(19) United States
(12) Patent Application Publication
(10) Pub. No.: US 2013/0261090 A1
(43) Pub. Date: Oct. 3, 2013

(54) METHOD FOR TREATMENT OF SORES IN THE ORAL CAVITY WITH A COMPOSITION CONTAINING ULTRAVIOLET LIGHT-ABSORBING INGREDIENTS

(71) Applicant: Beny RUB, Golden Beach, FL (US)

(72) Inventor: Beny RUB, Golden Beach, FL (US)

(21) Appl. No.: 13/832,551

(22) Filed: Mar. 15, 2013

Related U.S. Application Data
(60) Provisional application No. 61/619,087, filed on Apr. 2, 2012.

(51) Int. Cl. A61K 31/618; A61K 31/12; A61K 31/277

(52) U.S. Cl. CPC A61K 31/618; A61K 31/277; A61K 31/12
USPC 514/160

(57) ABSTRACT

The invention provides a method for treating ulcerations of the oral cavity, such as canker sores, sores associated with hand, foot, and mouth disease (HFMD), and sores associated with herpangina, with compositions containing ultraviolet light-absorbing ingredients. The ultraviolet light-absorbing ingredients include avobenzone, homosalate, octisalate, octocrylene, and/or oxybenzone. These treatments accelerate the healing process of the ulcerations of the oral cavity.

### CROSS REFERENCE TO RELATED APPLICATIONS

This application claims the benefit of priority to U.S. Provisional Patent Application No. 61/619,087, filed on Apr. 2, 2012, the content of which is hereby incorporated by reference in its entirety.

### FEDERAL SPONSORSHIP

This work was not federally sponsored research or development.

### FIELD OF THE INVENTION

The invention generally relates to using compositions containing ultraviolet light-absorbing ingredients for therapeutic purposes, particularly to using compositions containing ultraviolet light-absorbing ingredients to treat ulcerations of the oral cavity, and most particularly to using compositions containing avobenzone, homosalate, octisalate, octocrylene, and/or oxybenzone for treating canker sores, sores relating to hand, foot, and mouth disease (HFMD), and/or sores relating to herpangina to accelerate the healing process of these sores.

### BACKGROUND

Canker sores (mouth ulcers) are painful ulcers or open sores of the oral cavity. These sores manifest as ulcerations which appear on the tongue and/or inside lining of the lips, cheeks, and throat (mucosal membrane). The precise cause of the sores remains unclear, but multiple factors are suspected to result in outbreaks; these factors including tissue injury, food sensitivities, genetics, immune factors, bacterial infections, and emotional stress. Canker sores usually resolve without treatment in one to two weeks. See “The Difference Between Canker Sores and Cold Sores” dated Feb. 18, 2009 and accessed from “The Healthier Life” website on Feb. 22, 2012.

Sores similar to canker sores are symptoms of hand, foot, and mouth disease (HFMD), a common viral illness in children caused by an enterovirus, such as the coxsackie virus. HFMD most often occurs in toddlers and preschool age children, although adults and children of any age can contract this disease.

The first signs of HFMD generally include, fever, sore throat, and loss of appetite. Within a couple days after the appearance of the first signs, the infected child/adult will develop painful sores on the tongue, gums, and/or inside of the cheeks and rashes on the palms of the hands and/or soles of the feet. The sores begin as small red spots that blister and become ulcerated. HFMD usually resolves without treatment in one to two weeks.

Herpangina is another common viral illness in children also caused by an enterovirus. The symptoms of herpangina are fever, loss of appetite, and painful oral sores/lesions. These sores are similar are similar to those of HFMD, with the exception of their location. The sores of HFMD are generally located in the front of the oral cavity and those sores of herpangina are generally located in the back of the oral cavity and throat. Again, similar to HFMD, herpangina is a self-limited infection and usually resolves without treatment.

Although canker sores, sores associated with hand, foot, and mouth disease (HFMD), and sores associated with herpangina are not considered medically serious and usually resolve themselves without treatment in about two weeks time, these sores are painful and/or annoying and can make eating/drinking difficult, especially for children. Thus, treatments which accelerate the healing process would be very beneficial to children and adults suffering from these sores.

The above information regarding canker sores, hand, foot, and mouth disease, and herpangina can be found at the web sites of WedMD, Baby Center LLC, and Medicine Net.

Citation or identification of any reference/document in the instant application is not and should not be interpreted as an admission that such reference/documents is available as prior art to the present invention.

### SUMMARY OF THE INVENTION

The invention generally provides methods for treating mouth sores. More particularly, the invention provides methods for treating ulcerations of the oral cavity using compositions containing ultraviolet-light absorbing ingredients and further provides methods for using compositions containing ultraviolet light-absorbing ingredients to treat ulcerations of the oral cavity.

In one embodiment, the invention provides a method for treating a sore in the oral cavity of a subject by topically administering a composition including at least one ultraviolet light-absorbing ingredient to the sore in an amount sufficient to cover the sore.

The sore to be treated can be any type of sore, but preferably is a sore of the oral cavity, such as a canker sore, a sore associated with associated with hand, foot, and mouth disease (HFMD), or a sore associated with herpangina.

A “sore of the oral cavity” as used herein, can include, but is not limited to, sores appearing on the lips, on the inside of the lips, on the inside of the cheeks, on the tongue, in the throat, on the palate, on any oral mucosal membrane, and/or on the face in the skin around the mouth.

As used herein, the term “sore associated with hand, foot, and mouth disease” is a sore which manifests as a symptom of hand, foot, and mouth disease (HFMD).

As used herein, the term “sore associated herpangina” is a sore which manifests as a symptom of herpangina.

As used herein, the terms “treat” and “treating” refer to caring for a sore by decreasing and/or eliminating pain and discomfort caused by the sore and/or increasing the speed of healing and/or resolution of the sore. A treatment can include one or multiple applications of the composition to the sore(s). Additional applications can be immediately subsequent to the initial application or can be administered after a period of time passes from the initial administration of the composition; for example every four hours. The treatment accelerates the healing process and shortens the length of the healing time for a sore.

As used herein, a “subject”, “person”, and/or “patient” refers to a human being or an animal being in need of treatment for a sore.

As used herein, the term “ultraviolet light” refers to energy “beyond violet” in the spectrum. Ultraviolet light is emitted by the sun and by tanning beds and has both a shorter wavelength (<4000 angstroms) and a higher energy than visible light.

As used herein, the term “oral cavity” refers to the mouth and to the cavity of the mouth of a human or an animal as defined by jawbones.

As used herein, the term “mucosal membrane” refers to the epithelial lining of the mouth which is exposed to the external environment.

As used herein, the term “topical administration” refers to application of a product and/or composition to an external part of the body and/or to application only on a surface. In the instant invention, topical administration is emphasized since the ultraviolet light-absorbing ingredients used in the method can have adverse effects if taken internally into the body of the subject.

As used herein, the phrase “in an amount sufficient to cover the sore” refers to an amount of a composition necessary to coat the surface area of the sore. It is preferred to use the smallest amount necessary as the ultraviolet light-absorbing ingredients are potentially poisonous and thus, have undesirable side effects if taken internally in large amounts.

As used herein, the term “ingredient” refers a part of a mixture or a component of a mixture.

As used herein, the term “ultraviolet light-absorbing ingredients” refers to any ingredient in a composition which absorbs or blocks ultraviolet light, primarily ultraviolet light as radiated by the sun. The ultraviolet light-absorbing ingredients particularly contemplated for inclusion in the composition used in the described methods include avobenzone, homosalate, octisalate, octocrylene, and/or oxybenzone.

In another embodiment, the invention provides a method for treating a sore in the oral cavity of a subject by topically administering a composition including one or more of avobenzone, homosalate, octisalate, octocrylene, and oxybenzone to the sore in an amount sufficient to cover the sore.

It is contemplated that avobenzone, homosalate, octisalate, octocrylene, and oxybenzone can be included in the composition used in the described methods in any combination and/or any in amount.

A preferred composition for use with the described methods includes avobenzone, homosalate, octisalate, and octocrylene in percentages of 3% avobenzone, 10% homosalate, 5% octisalate, and 10% octocrylene.

Another preferred composition for use with the described methods includes avobenzone, homosalate, octisalate, octocrylene, and oxybenzone in percentages of 3% avobenzone, 13% homosalate, 5% octisalate, 5% octocrylene, and 6% oxybenzone.

Accordingly, it is an objective of the invention to provide new techniques of caring for and/or curing mouth sores.

It is another objective of the invention to provide new techniques for alleviating the discomfort caused by mouth sores.

It is another objective of the invention to provide new topical treatments for mouth sores.

It is still another objective of the invention to provide new techniques for accelerating the healing process of mouth sores.

It is yet another objective of the invention to provide new techniques of caring for and/or curing canker sores, sores associated with hand, foot, and mouth disease (HFMD), and sores associated with herpangina.

It is another objective of the invention to provide new techniques of alleviating the discomfort caused by canker sores, sores associated with hand, foot, and mouth disease (HFMD), and sores associated with herpangina.

It is still another objective of the invention to provide new topical treatments for canker sores, sores associated with hand, foot, and mouth disease (HFMD), and sores associated with herpangina.

It is yet another objective of the invention to provide new techniques for accelerating the healing process of canker sores, sores associated with hand, foot, and mouth disease (HFMD), and sores associated with herpangina.

It is another objective of the invention to use compositions containing ultraviolet light-absorbing ingredients for therapeutic purposes.

It is another objective of the invention to use compositions containing ultraviolet light-absorbing ingredients as topical therapeutics in the oral cavity.

It is yet another objective of the invention to use compositions containing ultraviolet light-absorbing ingredients to treat mouth sores and/or ulcerations of the oral cavity.

It is another objective of the invention to use compositions containing ultraviolet light-absorbing ingredients to treat canker sores, sores associated with hand, foot, and mouth disease (HFMD), and/or sores associated with herpangina.

It is yet another objective of the invention to use compositions containing avobenzone, homosalate, octisalate, octocrylene, and/or oxybenzone for treating treat mouth sores and/or ulcerations of the oral cavity.

It is yet another objective of the invention to use compositions containing avobenzone, homosalate, octisalate, octocrylene, and/or oxybenzone for treating canker sores, sores associated with hand, foot, and mouth disease (HFMD), and/or sores associated with herpangina.

It is still another objective of the invention to use compositions including avobenzone, homosalate, octisalate, and octocrylene in percentages of 3% avobenzone, 10% homosalate, 5% octisalate, and 10% octocrylene to treat mouth sores and/or ulcerations of the oral cavity.

It is still another objective of the invention to use compositions including avobenzone, homosalate, octisalate, and octocrylene in percentages of 3% avobenzone, 10% homosalate, 5% octisalate, and 10% octocrylene to treat canker sores, sores associated with hand, foot, and mouth disease (HFMD), and/or sores associated with herpangina.

It is yet another objective of the invention to use compositions including avobenzone, homosalate, octisalate, octocrylene, and oxybenzone in percentages of 3% avobenzone, 13% homosalate, 5% octisalate, 5% octocrylene, and 6% oxybenzone to treat mouth sores and/or ulcerations of the oral cavity.

It is yet another objective of the invention to use compositions including avobenzone, homosalate, octisalate, octocrylene, and oxybenzone in percentages of 3% avobenzone, 13% homosalate, 5% octisalate, 5% octocrylene, and 6% oxybenzone to treat canker sores, sores associated with hand, foot, and mouth disease (HFMD), and/or sores associated with herpangina.

Although the invention is described herein as embodied in a method for using compositions containing ultraviolet light-absorbing ingredients to treat mouth sores/ulcerations of the oral cavity, it is, nevertheless, not intended to be limited to the details described because various modifications and structural changes may be made therein without departing from the spirit of the invention.

Thus, the above embodiments, features, and objectives are merely exemplary of the invention. Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 shows the structural formula for avobenzone; IUPAC name, 1-(4-Methoxyphenyl)-3-(4-tert-butylphenyl)propane-1,3-dione.

FIG. 2 shows the structural formula for homosalate; IUPAC name, 3,3,5-Trimethylcyclohexyl 2-hydroxybenzoate.

FIG. 3 shows the structural formula for octisalate; IUPAC name, 2-ethylhexyl 2-hydroxybenzoate.

FIG. 4 shows the structural formula for octocrylene; IUPAC name, 2-ethylhexyl 2-cyano-3,3-diphenyl-2-propenoate.

FIG. 5 shows the structural formula for oxybenzone; IUPAC name, (2-hydroxy-4-methoxyphenyl)-phenylmethanone.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described methods, techniques, compositions, products, kits, and/or any further application of the principles of the invention as described herein, are contemplated as would normally occur to one skilled in the art to which the invention relates.

Painful mouth sores are often a symptom of viral illness in children; such as those sores which appear with hand, foot, and mouth disease (HFMD) and with herpangina. Although they are rarely considered serious and resolve without treatment, these sores decrease quality of life for the sufferer (of the sores) by being painful and annoying. For example, the sores associated with herpangina often appear in the back of the oral cavity and throat, thus making eating and drinking difficult.

The present invention was made, quite unexpectedly, when the inventor discovered that oral sores covered with a composition including ultraviolet light-absorbing ingredients disappeared within 24 to 48 hours. Thus, the invention offers a new method for managing the pain and discomfort resulting from oral sores by significantly decreasing the time required for healing as compared to time required for healing of sores left untreated.

Compositions including ultraviolet light-absorbing ingredients are commercially available as sunscreen or sunblock. Sunscreen/sunblock is a preparation used to protect exposed skin from the ultraviolet rays (UVA and UVB) of the sun and usually contains one or two types of active ingredients; inorganic particles and organic compounds. The inorganic particles, such as titanium dioxide or zinc oxide, form a physical barrier on the skin which reflects and scatters the ultraviolet rays. Organic compounds, such as avobenzone, homosalate, octisalate, octocrylene, and oxybenzone, absorb the ultraviolet rays and release the energy as heat.

Although protection of skin from harmful ultraviolet rays of the sun is the designated purpose of sunscreen, it has been used as a prophylactic against cold sores. For example, it is known that the sunscreen in Chapstick® (lip balm) can prevent cold sores from forming on the lips. See The Gainesville Sun, Sunday, Oct. 3, 1991.

The terms “cold sore” and “canker sore” are often used interchangeably, however, they are separate conditions; i.e. cold sores are not canker sores. They (cold sores) are groups of painful, fluid-filled blisters that appear on the lips or on the skin around the lips. Cold sores are contagious and almost always caused by the herpes simplex type-1 virus. See “The Difference Between Canker Sores and Cold Sores” dated Feb. 18, 2009 and accessed from “The Healthier Life” website on Feb. 22, 2012.

The method described herein is the first therapeutic use of compositions including ultraviolet light-absorbing ingredients for treatment of sores of the oral cavity. Although any ultraviolet light-absorbing ingredient is contemplated for use in the composition used in the method, avobenzone, homosalate, octisalate, octocrylene, and oxybenzone are particularly preferred.

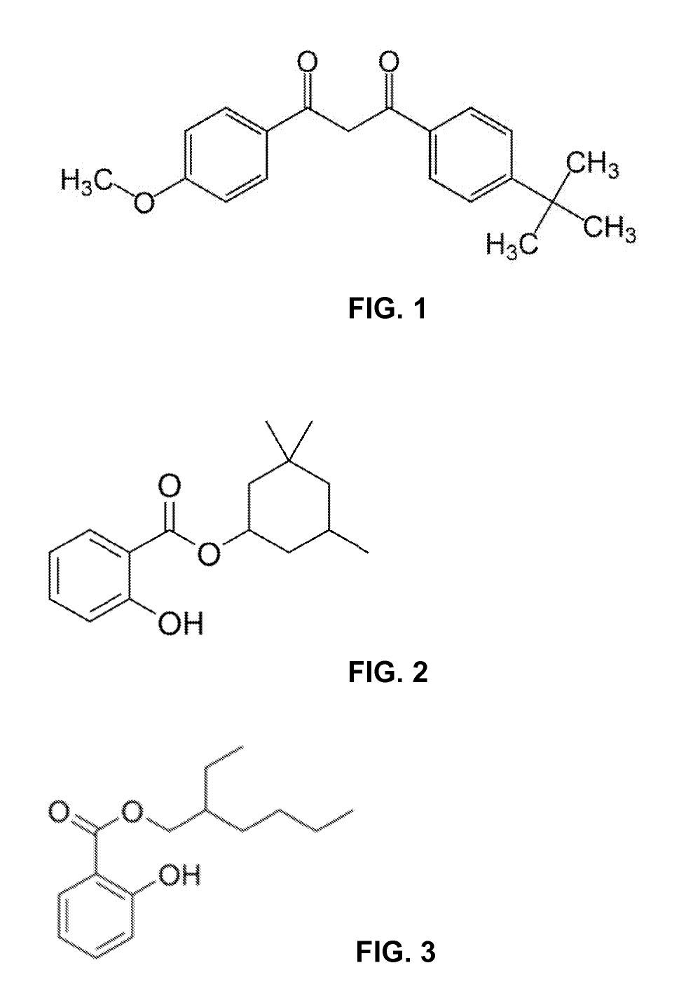

Avobenzone (FIG. 1), IUPAC name, 1-(4-Methoxyphenyl)-3-(4-tert-butylphenyl)propane-1,3-dione, is an oil-soluble dibenzoylmethane ingredient used in sunscreen products to absorb the full spectrum of ultraviolet A (UVA) rays.

Homosalate (FIG. 2), IUPAC name, 3,3,5-Trimethylcyclohexyl 2-hydroxybenzoate, is a cyclohexanol derivative used as an ingredient in sunscreen products to absorb ultraviolet B (UVB) rays.

Octisalate (FIG. 3), IUPAC name, 2-ethylhexyl 2-hydroxybenzoate, is an ester formed by the condensation of a salicylic acid with 2-ethylhexanol and used as an ingredient in sunscreen products to absorb ultraviolet B (UVB) rays.

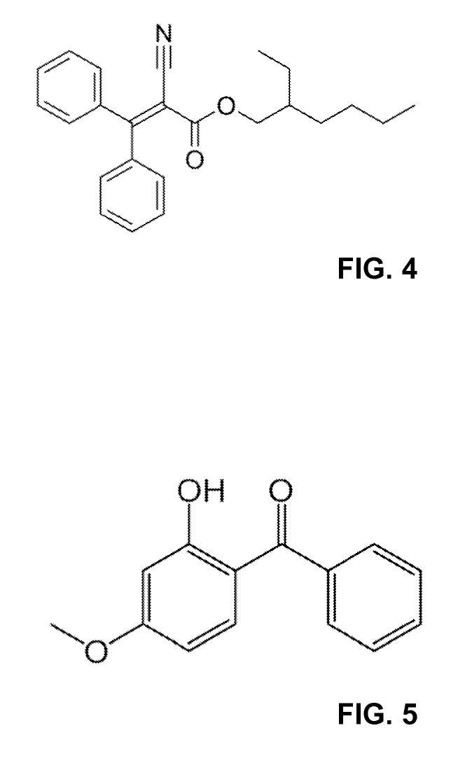

Octocrylene (FIG. 4), IUPAC name, 2-ethylhexyl 2-cyano-3,3-diphenyl-2-propenoate, is an ester formed by the condensation of a diphenylcyanoacrylate with 2-ethylhexanol and used as an ingredient in sunscreen products to absorb ultraviolet B (UVB) rays and short ultraviolet A (UVA) rays.

Oxybenzone (FIG. 5), IUPAC name, (2-hydroxy-4-methoxyphenyl)-phenylmethanone, is one of the benzophenone group of chemicals and is used as an ingredient in sunscreen products to both absorb ultraviolet B (UVB) rays and ultraviolet A (UVA) rays.

The descriptions and chemical structures of avobenzone, homosalate, octisalate, octocrylene, and oxybenzone can be found online at the Wikipedia website.

Any commercially-available sunscreen and/or sunblock is contemplated for use in the composition used in the described method.

### EXAMPLE 1

A sport cream sunscreen made by Coppertone® including active ingredients avobenzone, homosalate, octisalate, and octocrylene in percentages of 3% avobenzone, 10% homosalate, 5% octisalate, and 10% octocrylene.

### EXAMPLE 2

Coppertone® Sport Sunscreen Lotion (SPF 50) including active ingredients avobenzone, homosalate, octisalate, octocrylene, and oxybenzone in percentages of 3% avobenzone, 13% homosalate, 5% octisalate, 5% octocrylene, and 6% oxybenzone.

Inactive Ingredients: Water, Styrene Acrylates Copolymer, Propylene Glycol, Neopentyl Glycol Diheptanoate, Ethylenediamine/Neopentyl Glycol, Stearyl Hydrogenated Dimer Dilionleate Copolymer, Benzyl Alcohol, Acrylates/C10 30 Alkyl Acrylate Crosspolymer, Acrylates Crosspolymer, Triethanolamine, Chlorphenesin, Oleth 3, Tocopheryl Acetate (Vitamin E), Fragrance, Disodium EDTA

### Caution: Sunscreen/Sunblock Poisoning

As noted above, topical administration is imperative since the ultraviolet light-absorbing ingredients in the composition used in the method are potentially poisonous and can have adverse effects if taken internally into the body of the subject. The compositions must not be swallowed. Thus, when administering the composition only the smallest amount necessary to cover the sore should be used.

Sunscreen poisoning can occur when someone accidentally or intentionally swallows sunscreen.

Poisonous Ingredients: Older sunscreens used para-aminobenzoic acid (PABA) to protect skin from the sun's rays. Many of the currently-available sunscreens are PABA free but contain a number of other potentially poisonous ingredients such as cinnamates, padimate-O, salicylates (asprin-like compounds), and zinc oxide.

Symptoms: Sunscreens are generally considered non-poisonous/non-toxic for topical use. Most symptoms are caused by mild allergic reactions, eye irritation, and/or skin irritation. Eye irritation may occur after the eyes are exposed to the sunscreen. An allergic reaction may manifest as a rash on the skin.

More serious symptoms result from internalization of the sunscreen. Gastrointestinal irritation may manifest as nausea and vomiting. Allergic reactions may manifest in the lungs as shortness of breath and/or slowed respirations (extremely high doses).

Home Care: When someone swallows sunscreen/sunblock seek immediate medical help. Do not make the person vomit unless told to do so by Poison Control or a health care professional. Before calling emergency determine the patient's age, weight, and condition, name of the product swallowed and its ingredients, the time the product was swallowed, and the amount of product that was swallowed.

Poison Control: The National Poison Control Center (1-800-222-1222) can be called from anywhere in the United States. This national hotline number will connect you to experts in poisoning. The experts will give you further instruction.

This is a free and confidential service. All local Poison Control Centers in the United States use this national number. You should call if you have any questions about poisoning or poison prevention. It does not have to be an emergency situation. You can call for any reason, 24 hours a day, 7 days a week.

Emergency Room: The health care professional will measure and monitor the patient's vital signs, including temperature, pulse, breathing rate, and blood pressure. Symptoms will be treated as appropriate. The treatments can include breathing support, fluids through a vein (IV), and/or a tube through the mouth into the stomach to flush out the stomach.

Prognosis: How well the patient does depends on the amount of poison swallowed and how quickly treatment is received. The faster you get medical help, the better the chances for recovery.

Swallowing sunscreen/sunblock usually causes only mild stomach upset and vomiting. Some sunscreens/sunblock may contain ethanol (ethyl alcohol). Children who swallow a large amount of such products may become drunk (intoxicated). Additionally, swallowing a large amount of sunscreen/sunblock made from salicylates could cause a condition similar to aspirin overdose.

### REFERENCES

McGee, D. L. “Local and Topical Anesthesia.” In: Roberts, J. R and Hedges, J. R., editors, Clinical Procedures in Emergency Medicine, Chapter 29, 5^{th} Edition, Saunders Elsevier, Philadelphia, Pa. 2009.

Kerr, F. “Salicylates” In: Shannon, M. W. Borron, S. W., and Burns, M. J. editors, Haddad and Winchester's Clinical Management of Poisoning and Drug Overdose, Chapter 48, 4^{th} edition, Saunders Elsevier, Philadelphia, Pa. 2007. Updated on Jan. 27, 2010 by Jacob L. Heller, MD, MHA, Emergency Medicine, Virginia Mason Medical Center, Seattle, Wash. Also reviewed by David Zieve, MD, MHA, Medical Director, A.D.A.M., Inc.
*The above-information regarding poisoning is for information only and not for use in the treatment and/or management of an actual exposure to poison. If you have an exposure, you should call your local emergency number (911) and/or the National Poison Control Center at 1-800-222-1222.

All patents and publications mentioned in this specification are indicative of the level of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, formulations, methods, techniques, systems, programs, and kits described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

What is claimed is:

1. A method for treating a sore in an oral cavity of a subject comprising providing a composition including at least one ultraviolet light-absorbing ingredient and topically administering the composition to the sore in an amount sufficient to cover the sore.

2. The method according to claim 1, further comprising a second topical administration of the composition, the second topical administration administered immediately subsequent to a first administration or after a period of time has passed since the first administration.

3. The method according to claim 1, wherein the sore is a canker sore, a sore associated with hand, foot, and mouth disease (HFMD), or a sore associated with herpangina.

4. The method according to claim 1, wherein the at least one ultraviolet light-absorbing ingredient is selected from the group consisting of avobenzone, homosalate, octisalate, octocrylene, and oxybenzone.

5. A method for treating a sore in an oral cavity of a subject comprising providing a composition including one or more of avobenzone, homosalate, octisalate, octocrylene, and oxybenzone and topically administering the composition to the sore in an amount sufficient to cover the sore.

6. The method according to claim 5, further comprising a second topical administration of the composition, the second topical administration administered immediately subsequent to a first administration or after a period of time has passed since the first administration.

7. The method according to claim 5, wherein the sore is a canker sore, a sore associated with hand, foot, and mouth disease (HFMD), or a sore associated with herpangina.

8. The method according to claim 5, wherein the composition includes avobenzone, homosalate, octisalate, and octocrylene in percentages of 3% avobenzone, 10% homosalate, 5% octisalate, and 10% octocrylene.

9. The method according to claim 5, wherein the composition includes avobenzone, homosalate, octisalate, octocrylene, and oxybenzone in percentages of 3% avobenzone, 13% homosalate, 5% octisalate, 5% octocrylene, and 6% oxybenzone.
